(19) United States
(12) Patent Application Publication
(10) Pub. No.: US 2026/0199677 A1
(43) Pub. Date: Jul. 16, 2026

(54) A DEVICE FOR ENHANCED WOUND HEALING AND METHODS FOR TRACKING THE WOUND CONDITION THEREOF

(71) Applicant: ENNOCURE MEDTECH LTD, Maor (IL)

(72) Inventor: Manuela HOD, Maor (IL)

(21) Appl. No.: 19/135,270

(22) PCT Filed: Dec. 3, 2023
(86) PCT No.: PCT/IL2023/051237

§ 371 Date: Jun. 3, 2025

Related U.S. Application Data
(60) Provisional application No. 63/385,973, filed on Dec. 4, 2022.

(51) Int. Cl. A61N 1/36; A61F 13/00; A61F 13/01; A61L 15/22; A61N 1/04

(52) U.S. Cl. CPC A61N 1/36031; A61F 13/00051; A61F 13/01046; A61L 15/22; A61N 1/0468; A61N 1/0492; A61F 2013/00246

(57) ABSTRACT

A multi-layered dressing device is provided to be placed on a wound. The multi-layered dressing device comprises a dressing layer to be placed on the wound while extended slightly beyond the perimeter of the wound, at least one sensor in the dressing layer for transmitting electronic signals from a vicinity of the wound; at least two electrodes in the dressing layer one of which is positioned externally to the perimeter of the wound, wherein the electrodes are capable of transmitting stimulating signals to the wound, and a PCB layer for activating the sensors and the electrodes. The PCB layer accommodates a computing device configured to receive the electronic signals and control the stimulating signals. A method of tracking and enhancing the condition of the wound through the dressing device is also provided.

### TECHNICAL FIELD

The present disclosed subject matter relates to wound healing. More particularly, the present disclosed subject matter relates to a device for enhanced wound healing as well as tracking the healing condition.

### BACKGROUND

The healing of wounds is a complex process that ultimately should lead to wound closure. A variety of factors can delay the wound healing process and lead to chronic wounds, as an example. These types of wounds have failed to finalize the healing process within less than 42 days. Factors that influence the healing process are qualified by local factors as well as systematic factors that may include age and gender, sex hormones, general life factors such as stress, alcoholism, smoking and nutrition. Moreover, the individual health condition affects the wound healing processes. Diseases such as diabetes, keloids, fibrosis, hereditary healing disorders, jaundice, and uremia have been proven to interfere with the healing process.

Among the above listed diseases, diabetes is the most common disease. Globally, an estimated number of 422 million adults were living with diabetes in 2014, of which 15% are estimated to develop diabetic foot ulcers (DFU). The non-healing wounds condition of DFUs is a serious complication of diabetes that result, in 84% of the patients, in lower leg amputations (Brem and Tomic-Canic, 2007).

In fact, chronic wounds—wounds that can take years to heal and often remain in the inflammatory phase—are a challenge for both, patients and health care systems. Treatment of chronic wounds is time-consuming and expensive for both the patients and the healthcare system. Routine replacement of the dressing/bandage is one of the common treatment methods for such cases. Simple dressings need to be exchanges several times per day while advanced wound dressing can remain in place up to 5 days. Patients must routinely attend checks in a local wound center, hospital, or personal home-care to analyze current wound status and obtain advice on further treatment. In severe wound cases, the patient gets a local treatment at the wound clinic or is further transferred for advanced treatments.

Due to the massive complexity of wound care, advanced wound care devices and methods are being employed among which are wound dressings and wound therapy devices. Ideal conditions should be maintained in order to enhance wound healing. Generally, ideal wound healing is obtained by keeping the wound moist and oxygenated, preventing bacterial infection, and closely monitoring the progress so as to identify complications and adjust treatment as required.

Invasive and non-invasive methods are used for wound treatment, and they are commonly applied on both acute and chronic wounds. Invasive methods include skin substitutes and debridement, which can be expensive, time-consuming, and slow in showing any positive effect. In common practice, wounds are treated non-invasively with wound dressings and ultrasound, negative pressure wound therapy, hyperbaric oxygen therapy and electric stimulation.

Wound assessment methods are part of the standard wound care process. The wound's physical dimensions are measured manually or using a camera and then, documented. In case of suspected wound infection, the caregiver will advise a biopsy to be performed or use advanced assessment cameras during the assessment process in order to acquire more accurate information.

The wound condition documentation and consequently the tracking of the wound healing progress is traditionally performed manually by the caregiver. In a more modern environment, automatic and digital systems are used for documentation and statistical analysis of the progress of wound healing or deterioration.

Wound assessment methods and corresponding dressings are needed in order to automatically measure the wound parameters for the assessment of its condition.

### BRIEF SUMMARY

According to a first aspect of the present disclosed subject matter, there is a need to provide a comprehensive device for effectively treating wounds while simultaneously monitoring the healing progress.

According to another aspect of the present subject matter, electronic elements such as sensors and electrodes are embedded within a dressing so as to obtain data on the wound condition and allow control over the healing progress.

According to yet another object of the present subject matter, a method is provided that enables both: treatment of a wound by means of the dressing and continues monitoring of the wound condition.

It is therefore provided, in accordance with an embodiment of the present subject matter a multi-layered dressing device to be placed on a wound having a perimeter, the multi-layered dressing device comprising:
a bioelectric layer to be placed on the wound slightly beyond the perimeter;
at least one sensor in the bioelectric layer capable of transmitting electronic signals from a vicinity of the wound;
at least two electrodes in the bioelectric layer wherein one of the at least two electrodes is positioned externally to the perimeter of the wound, wherein the at least two electrodes are capable of transmitting stimulating signals to the wound, and
a PCB layer for activating the at least one sensor and the at least two electrodes, wherein the PCB layer accommodates a computing device configured at least to receive the electronic signals and control the stimulating signals.

In accordance with another embodiment, the bioelectric layer comprises a polymer layer to be in an intimate contact with the wound, and a gas permeable layer placed on top of the polymer layer, wherein the gas permeable layer has an edge that extend beyond the perimeter of the wound.

In accordance with another embodiment, at least one of the at least one sensor is positioned in the polymer layer.

In accordance with another embodiment, the at least two electrodes are placed within the gas permeable layer.

In accordance with another embodiment, the at least two electrodes are placed beyond the perimeter of the wound.

In accordance with another embodiment, the gas permeable layer is provided with liquid reservoirs.

In accordance with another embodiment, the liquid reservoirs are microfluid channels that are gas permeable but liquid impermeable and wherein the microfluid channels are configured to transfer O₂ or H⁺ to an environment of the wound.

In accordance with another embodiment, the O₂ or H⁺ are generated within the microfluid channels.

In accordance with another embodiment, one of the at least two electrodes is configured to stimulate the generation of O₂ or H⁺ in the microfluid channels.

In accordance with another embodiment, the PCB layer is further provided with an interface allowing a user to interact with the computing device.

In accordance with another embodiment, the PCB layer is further provided with a signal generator capable of transmitting information from the computing device to an external device.

In accordance with another embodiment, the PCB layer is further provided with a display for displaying information gathered from the at least one sensor.

In accordance with another embodiment, the PCB layer is stacked on top of the bioelectric layer.

In accordance with another embodiment, the PCB layer is spaced apart from the bioelectric layer.

It is therefore provided in accordance with another embodiment of the present subject matter, a method of tracking and enhancing a wound condition comprising:
providing a sensor-contained bioelectric wound dressing;
placing the bioelectric wound dressing on the wound, slightly beyond a perimeter of the wound;
providing a layer of electrodes wherein at least one of the electrodes is positioned beyond the perimeter of the wound;
providing a PCB layer having a power source so as to power at least the sensors and the electrodes;
providing a controller configured to receive signals from the sensors and transmit signals from the electrodes.

In accordance with another embodiment, the method further comprises:
providing a gas permeable layer having liquid reservoirs and stacking it on top of the sensors-contained bioelectric layer;
inserting liquid to within the liquid reservoirs;
initiating generation of oxygen and/or ionic hydrogen in the liquid reservoirs using signals from the electrodes.

In accordance with another embodiment, the method further comprises:
providing an interface in the PCB layer;
interacting with the sensors and electrodes through the interface.

In accordance with another embodiment, the method further comprises providing a signal generator to the PCB so as to allow transferring information to external devices.

In accordance with another embodiment, the external devices can be a display.

In accordance with another embodiment, the interface comprises a display.

It is also provided in accordance with yet another embodiment of the present subject matter a system for enhancing wounds healing and tracking the wounds condition comprising:
a plurality of multi-layered dressing devices, and
at least one external device comprising an activation app configured to receive information from the at least one sensor and transfer information to the at least two electrodes.

Unless otherwise defined, all technical and scientific terms used herein have the same meaning as commonly understood by one of ordinary skill in the art to which this disclosed subject matter belongs. Although methods and materials similar or equivalent to those described herein can be used in the practice or testing of the present disclosed subject matter, suitable methods and materials are described below. In case of conflict, the specification, including definitions, will control. In addition, the materials, methods, and examples are illustrative only and not intended to be limiting.

### BRIEF DESCRIPTION OF THE DRAWINGS

Some embodiments of the disclosed subject matter described, by way of example only, with reference to the accompanying drawings. With specific reference now to the drawings in detail, it is stressed that the particulars shown are by way of example and for purposes of illustrative discussion of the preferred embodiments of the present disclosed subject matter only, and are presented in the cause of providing what is believed to be the most useful and readily understood description of the principles and conceptual aspects of the disclosed subject matter. In this regard, no attempt is made to show structural details of the disclosed subject matter in more detail than is necessary for a fundamental understanding of the disclosed subject matter, the description taken with the drawings making apparent to those skilled in the art how the several forms of the disclosed subject matter may be embodied in practice.

In the drawings:

FIG. 1 schematically illustrates a cross sectional view of a microelectronic multi-layered dressing device for enhanced wound healing, in accordance with some exemplary embodiments of the disclosed subject matter;

FIG. 2 illustrates a cross sectional view of a microelectronic multi-layered dressing device for enhanced wound healing, in accordance with some other exemplary embodiments of the disclosed subject matter;

FIG. 3 illustrates a cross sectional view of a microelectronic dressing device for enhanced wound healing with separated PCB layer, in accordance with some exemplary embodiments of the disclosed subject matter;

FIG. 4 illustrates a cross sectional view of a microelectronic multi-layered dressing device for enhanced wound healing, in accordance with some other exemplary embodiments of the disclosed subject matter;

FIG. 5 schematically illustrates the components of a microelectronic dressing device, in accordance with some exemplary embodiments of the disclosed subject matter;

FIG. 6 depicts an exploded view of a microelectronic multi-layered dressing device, in accordance with some exemplary embodiment of the disclosed subject matter;

FIG. 7 illustrates a flowchart diagram of a method of tracking and enhancing a wound healing process, in accordance with some exemplary embodiments of the disclosed subject matter;

FIG. 8 illustrates a flowchart diagram of a method of tracking and enhancing a wound healing process, in accordance with other exemplary embodiments of the disclosed subject matter, and

FIG. 9 schematically illustrates a block diagram of a system for tracking and enhancing wound healing process, in accordance with other exemplary embodiments of the disclosed subject matter.

FIG. 10 depicts images of the petri dishes after each of the timeslots showing the results of the in-vitro experiment made in accordance with exemplary embodiment of the disclosed subject matter.

### DETAILED DESCRIPTION

Before explaining at least one embodiment of the disclosed subject matter in detail, it is to be understood that the disclosed subject matter is not limited in its application to the details of construction and the arrangement of the components set forth in the following description or illustrated in the drawings. The disclosed subject matter is capable of other embodiments or of being practiced or carried out in various ways. Also, it is to be understood that the phraseology and terminology employed herein is for the purpose of description and should not be regarded as limiting. The drawings are generally not to scale. For clarity, non-essential elements were omitted from some of the drawings.

The terms “comprises”, “comprising”, “includes”, “including”, and “having” together with their conjugates mean “including but not limited to”. The term “consisting of” has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosed subject matter may be presented in a range format. It should be understood that the description in range format is merely for convenience and brevity and should not be construed as an inflexible limitation on the scope of the disclosed subject matter. Accordingly, the description of a range should be considered to have specifically disclosed all the possible sub-ranges as well as individual numerical values within that range.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination or as suitable in any other described embodiment of the disclosed subject matter. Certain features described in the context of various embodiments are not to be considered essential features of those embodiments, unless the embodiment is inoperative without those elements.

According to an aspect of the present subject matter, it is an object to provide a bioelectronic wound dressing that is placed on an open wound in order to monitor it and facilitate its healing.

It is another aspect of the present subject matter to provide a wound dressing that is in a shape of a patch type consumable that senses the wound size and presence of infection and inflammation in a self-sufficient manner.

According to yet another aspect of the present subject matter, the bioelectronic dressing includes a set of electrodes that, on one hand, generate an electric field or electric stimulating signals to accelerate the wound healing, and on the other hand, produce pure O₂ gas and release H⁺ (lowers the pH) that are delivered to the wound site through polymeric matrix channels and/or the walls of microchannel that are provided within the dressing material.

No external devices and no external gas delivery systems are required for the dressing. The electric field, stimulation and gas formation are simultaneously or separately occurring in a controlled manner. The bioelectronic dressing comprises sensors that provide constant feedback on wound condition, and therefore, facilitate a controlled and adjustable maintenance of substantially ideal conditions in the environment of the wound.

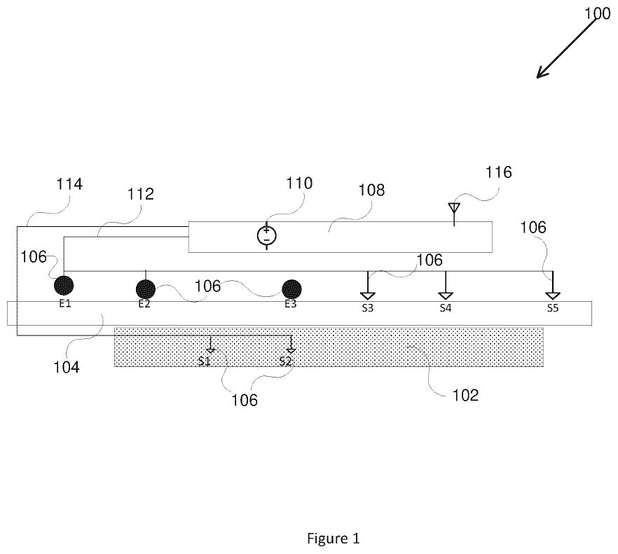

Referring now to FIG. 1 illustrating a cross sectional view of a microelectronic multi-layered dressing device for enhanced wound healing, in accordance with some exemplary embodiments of the disclosed subject matter.

The device 100 is a disposable device that is provided with several layers; hence a multi-layered device. A first layer that is configured to be in the closest proximity to and in an intimate contact with the wound is a dressing layer 102. The dressing layer 102 has several characteristics that ensure the progression of the wound healing process such as moisture control, mechanical protection of the wound, transmission of gasses, in some cases, elimination of infection and microorganisms development, low adherence to the skin despite the requirement for intimate contact, pain relief etc., and therefore, is preferably made of a polymeric layer selected from the group of polydimethylsiloxane (PDMS), polycaprolactone, polyvinylpyrrolidone, polyvinylalcohols, polyethylene glycols, poly(lactic-co-glycolic acid), carboxy cellulose and its derivatives, polysaccharides, other types of carbon-based hydrogels, a combination thereof, or the like.

Optionally, in accordance with another preferred embodiment, the dressing layer 102 can comprise active materials such as antibacterial and antiseptic components, in the form of nanoparticles, salts, or encapsulated drugs so as to support the wound healing process.

As mentioned herein before, the dressing is bioelectronic and comprises electronic elements 106, some of which are sensors (S1, S2) that are embedded within the dressing layer 102. The sensors S1 and S2 can receive information from the vicinity of the wound. Explanation on the functionality of the sensors will be provided herein after.

On top of the dressing layer 102, a gas permeable layer 104 is provided wherein the gas permeable layer 104 is configured to embed additional set of electronic elements 106 that can receive information from the vicinity of the wound. The gas permeable layer 104 is preferably made of materials selected from a group of materials such as polyurethane, PDMS, polyamides as Nylon, a combination thereof, or the like. Preferably, the gas permeable layer 104 has an edge that is beyond the perimeter of the dressing layer 102 and beyond the perimeter of the wound.

Additional plurality of electronic elements 106 is provided, preferably, on an upper surface of the gas permeable layer 104. The embedded electronic elements 106 in the dressing layers 102 are configured to receive information from the vicinity of the wound using sensors S1 and S2 that are embedded within the dressing layer 102. In this way, the wound environment is monitored while additional set of sensors S3, S4, and S5 is embedded on the upper surface of the gas permeable layer 104. An additional set of electronic elements 106, electrodes E1, E2 and E3, that can affect the healing process are also provided to the permeable layer 104; the electrodes affect the condition of the wound so as to facilitate the healing process. The electronic elements 106 can be embedded within the layers, connected to the layer's surface, adhered to the layer, or placed adjacent to the dressing using any other possible manner, without limiting the scope of the present subject matter.

As mentioned herein before, for monitoring purpose, the multi layered device 100 is provided with sensors S1-S5 that establish a sensing system. The wound sensing mechanism can be based on two types of parameters of the wound: physical parameters such as temperature, shape, or volume; and chemical wound parameters such as moisture, pH, oxygen level, and uric acid sensing. The wound sensing mechanism can be based on a pH sensor that will be embedded within the bioelectronic layer in closed proximity to the wound, however, it is preferable that it will comprise at least one additional sensor in at least one of the bioelectric layers, which are the dressing layer 102 and the gas permeable layer 104. The additional sensors are selected from a group of sensors such as pH sensor, temperature sensor, oxygen sensor, ureic acid sensor, bioimpedance, OH, a combination thereof, or the like. In this configuration, the bioelectric layers of the dressing are sensing the wound and its environment in a continuous and efficient manner. Other types of electronic elements 106 are the electrodes E1, E2, E3, some of which are provided adjacent to the wound dressing and some of which are located in other positioned of the device, as will be described hereinafter.

The electrodes E1, E2, E3 are a part of a circuit layer that preferably includes anodes and cathodes that are electrically connected using wires 112 with a current source 110 that is preferably provided on a printed circuit board (PCB) layer 108, which is a part of the device 100. The wires 112 are also connected to the sensors S3, S4, and S5 while the sensors S1 and S2 are connected to the current source 110 with wires 114. The anodes and cathodes are distributed over the gas permeable layer 104 in a predetermined architecture and geometry; however, at least one cathode (E3) is preferable positioned substantially in the center of the wound while at least one anode (E1) is preferably positioned in a vicinity of an intact portion of the skin (healthy skin), or vice versa. The current created in the current source 110 can be DC that is configured to generate an electric signal of about 0.001-2 mA.

The electrodes comprise similar conductors, selected from a group of conductors such as copper, silver, conductive polymers, a combination thereof or the like. According to another embodiment, the electrodes can also comprise electrochemical different materials selected from a group of materials such as graphene/Ag, Zn/Cu, Zn/Ag, Pt/Au, a combination thereof, or the like. Preferable metals for direct current approach are especially Zn and Ag as their ions have antiseptic and antibacterial properties; however, Pt has also suitable properties as electrode material.

Alternatively or additionally, AC current can be used and an adequate electrode design is configured to generate pulsed electric fields. The pulsed fields can have different voltage, current, pulse shape, and length. Most preferably is the use of low voltage pulsed current, wherein for this case, the location of the electrodes in the vicinity of the wound or the intact skin is of lower importance.

Alternatively or additionally, high voltage pulsed current can be used as well.

An electric signal through the wound can be generated between at least two of the electronic elements E1, E2, E3, S3, S4, and S5 that are embedded in the gas permeable layer 104.

Alternatively or additionally, at least two electrode elements can be placed on intact skin and pulsed current can be generated with different voltage, current, pulse shape, and length.

Electric currents of different shapes can be generated separately and simultaneously to activate at least one sensor operation.

It should be noted that although an example of five sensors and their distribution in the dressing are presented and three electrodes are shown in the example, this is merely an example and any number of electronic elements can be embedded within the bioelectronic layers without limiting the scope of the present subject matter. The number of sensors and electrodes as well as their distribution in the dressing device can be determined according to the characteristics of the wound itself, such as size and condition.

It should be noted that the power source can be selected from a group of power sources such as a single cell battery on the device, an energy receiving function to generate electric energy through wireless connection, a combination thereof, or the like.

In accordance with another embodiment, a signal generator 116 is provided on the PCB layer 108 that transfers the data retrieved from the sensors to a processor, a controller, or any other external device selected from a group of electronic devices such as computer software, health application, a combination thereof or the like that are provided on a personal computer, a tablet, a cellphone, a terminal or any other option through information transfer technology connection such as Wi-Fi. The signal generator 116, which can be selected from a group of signal transmitters such as RF generator, wave generator, function generator, a combination thereof or the like, transfers the signal of the electrodes E1, E2, E3 as well as the information gathered from the sensors S1, S2, S3, S4, S5 to external devices.

An external application system allows the external activation of different electric stimulation protocols and obtains the measured information from the sensor systems S1, S2, S3, S4, S5.

In software applications, the wound information is obtained, stored and analyzed continuously.

It should be mentioned that the PCB 108 can be either rigid or flexible. In the case a rigid PCB is used, it should occupy up to 20% of a total surface area of the device; however, if the PCB is flexible, its dimensions should be as large or slightly smaller than the surface area of the device, as shown in FIG. 1.

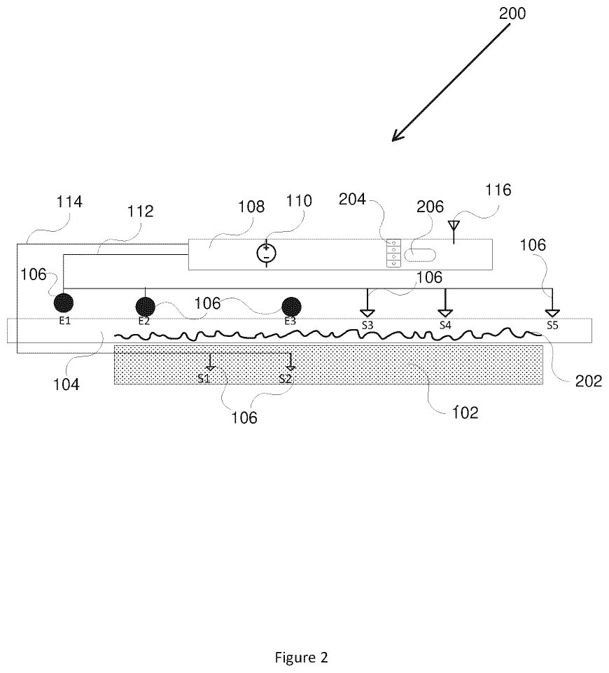

Reference is now made to FIG. 2 illustrating a cross sectional view of a microelectronic multi-layered dressing device for enhanced wound healing, in accordance with some other exemplary embodiments of the of the disclosed subject matter.

The dressing device 200 comprises similar components as device 100, however, the device 200 further comprises microfluid reservoirs configured to deliver essential gasses to the wound environment and facilitate its healing process.

The device 200 further comprises a plurality of microfluid channels 202 that are integrated within at least one of the dressing layer 102 and the gas permeable layer 104. The microchannels 202 that are present within the gas permeable layer 104 are provided with the same characteristics-they are made of a material that is gas permeable; however, is impermeable to liquids. The microchannels 202 are provided with liquid selected from a group of liquids such as water, liquid acid solution, hydrogen peroxide, a combination thereof or the like. The liquid can be inserted to within the microchannels 202 by injection, as an example, and is adapted to move freely within the channel. The microchannels 202 can be filled also with gas produced in the electrodes E1, E2, and E3 or produced within the channels themselves while the gas can penetrate through the microchannels walls to the dressing 102 and into the wound area.

Optionally or alternatively, the microfluids channels 202 can be filed with liquid or gases through pumps that are connected to a controller that controls the pumps operation upon a demand that accords the condition of the wound.

The electrodes E1, E2, and E3 that are in contact with the fluid that is flowing within the reservoirs in the microchannels 202 can react spontaneously to form oxygen. The liquid is stored and can flow within the reservoirs without leaking out to the layer in contact with the wound. The liquid in the microchannels can be transferred to one of the electrodes and generate a chemical reaction while generating O₂ or H⁺. The oxygen gas or hydrogen ions permeates outside the channel and saturates the wound with oxygen or hydrogen. This process can be repeatably controlled, and oxygen can be generated in one or several steps. Oxygen can be generated by chemical reaction or electrolysis. This process is controlled since the wound is monitored at all times using sensors SX (X denotes 1-5). The information from the sensors is gathered, processed, and is used in order to control the generation of the oxygen and hydrogen that in turn, is controlled by the electrodes.

Optionally and alternatively, a user can interact with the device 200 through an interface 204 that is preferably mounted on the PCB 108. The interface 204 allows the user to control the activation of the electrodes E1, E2, and E3, as an example, to activate at least one of the sensors S1 or S2 that are provided in the dressing layer 102 or at least one of the sensors S3, S4, and S5 that are provided in the gas permeable layer 104. Since the electronic components 106 are distributed within the layers in different orientations relative to the wound, the ability to control the individual electronic components 106 through the interface 204 can provide localized information and treatment that is more effective and efficient.

Optionally or alternatively, a display 206 is provided that enables the user to see parameters or elements that can be changed or controlled, as well as parameters that reflect the condition of the wound. This is also important for the efficiency of monitoring and healing the wound.

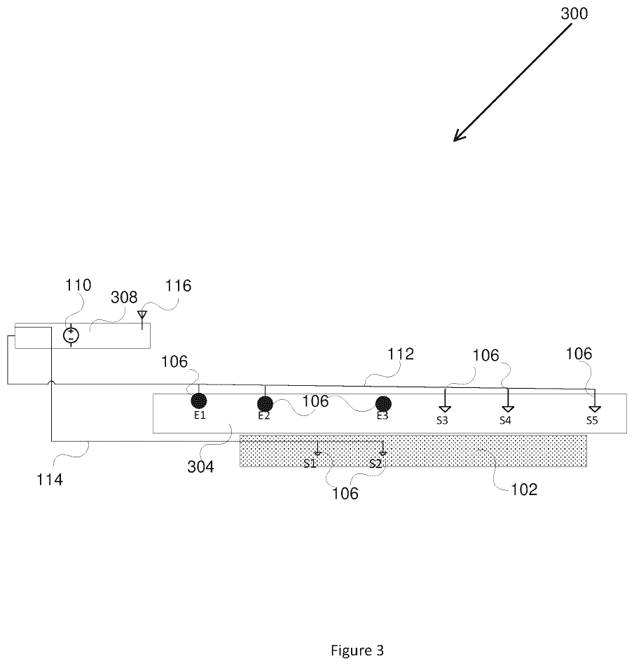

Referring now to FIG. 3 illustrating a cross sectional view of a microelectronic dressing device for enhanced wound healing with separated PCB layer, in accordance with some exemplary embodiments of the disclosed subject matter.

According to another embodiment, the dressing layer 102 with embedded sensors is substantially the same as the layers shown herein in the previous embodiments; however, on top of the dressing layer 102, a gas permeable layer 304 is provided in which additional electronic components 106 are provided; e.g. EX and SX. In this case, the electronic components 106 are embedded within the layer 304 rather than mounted on the surface of the layer. As mentioned herein before, the perimeter of the gas permeable layer 304 is larger than the perimeter of the dressing layer 102 so that a part of the perimeter of the gas permeable layer 304 is adhered to the healthy skin around the wound. The electronic components 106 are organized in the same manner as explained herein before.

The electronic components 106 are electrically connected to a power source 110 by wires 112 and 114 for their activation.

Optionally or additionally, a PCB layer 308 is provided and is electrically connected to the bioelectronic layers 102 and 304. The PCB layer 308 is not provided in the same stack as the bioelectronic layers, is separated from the gas permeable layer 304, and can be positioned in the vicinity of the bioelectronic layers. The PCB layer 308, as mentioned herein before, is provided with a power source 110 that activates electrically powered elements.

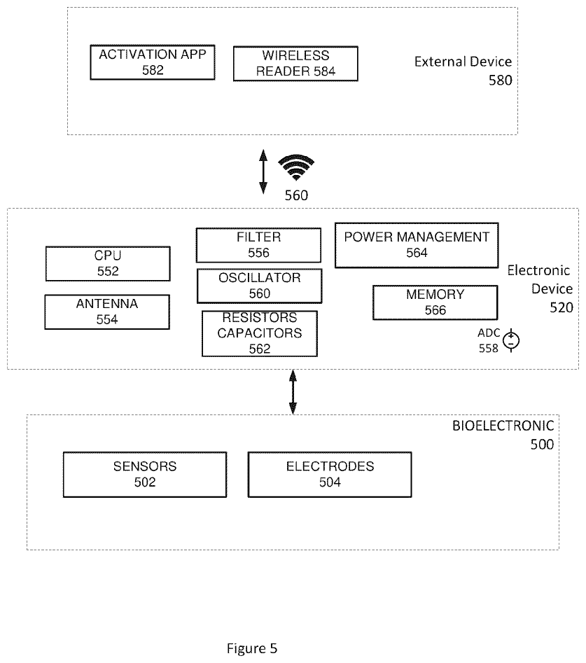

Optionally or additionally, the PCB layer 308 is provided with computing and controlling elements (shown in FIG. 5) that can receive or transmit signals from and to the sensors SX that are embedded within the dressing layer 102 and the gas permeable layer 304. Additionally, the computing and controlling elements can transmit signals to the wound or the skin through the electrodes EX in the gas permeable layer 304.

Optionally or additionally, an interface and/or display (not shown in this figure) can be incorporated within the PCB layer 308 as indicated herein before.

Optionally or additionally and in accordance with yet another embodiment of the present subject matter, liquid reservoir and/or microchannels can be formed in the gas permeable layer so as to transfer oxygen or hydrogen into the environment of the wound for healing purposes.

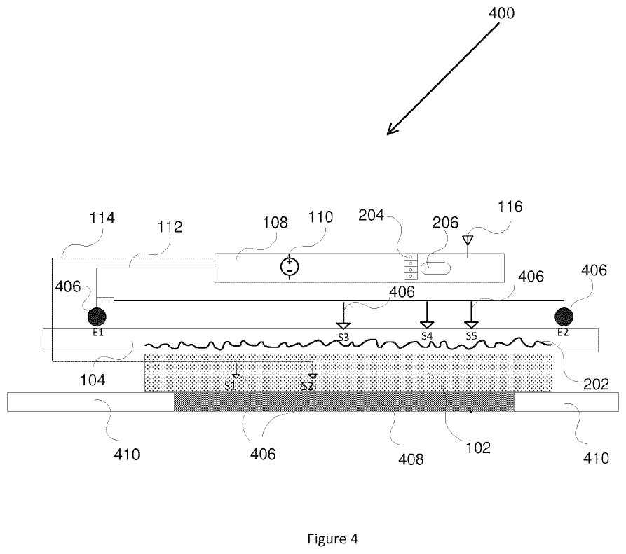

Reference is now made to FIG. 4 illustrating a cross sectional view of a microelectronic multi-layered dressing device for enhanced wound healing, in accordance with some other exemplary embodiments of the of the disclosed subject matter.

Microelectronic dressing device 400 comprises bioelectronic layers 102 and 104 provided with electronic elements 406 that are distributed within the layers in the following manner: S1 and S2, which are sensors mainly capable of sensing the environment, are embedded within the dressing layer 102, while additional sensors S3, S4, and S5 are mounted on the surface of gas permeable layer 104. All the sensors are mounted in the layers on top of the areas that are in the vicinity of the wound 408. The sensors in the dressing layer 102 are electrically connected by wires 114 to the power source 108 of the PCB 108, which is stacked as a top layer of the multi-layered device 400. The sensors in the gas permeable layer 104 are electrically connected by wires 112 as well to the power source 108 of the PCB 108.

The electrodes from the electronic elements 406 are organized in an architecture that is different from the architectures that are seen in the previous embodiments. At least two electrodes E1 and E2 are organized on the edges of the gas permeable layer 104 on areas that will be placed adjacent to the healthy skin 410 that surrounds the wound area 408, while positioned substantially opposite each other.

When current is transmitted through the electrodes (the anode and the cathode), electro-stimulation is transferred through the wound tissues 408, a process that facilitates the healing process.

Optionally or additionally, pure O₂ gas and H⁺ (lower pH) is delivered to the wound site 408 through polymeric matrix channels and/or the walls of microchannel 202 that are provided within the dressing material. The oxygen further promotes the healing process of the wound.

It should be noted that any architecture in which the multi-layered microelectronic dressing device that comprises polymer layers in which electrical elements that sense the wound condition and stimulate the wound tissues in a controlled manner is covered by the scope of the present subject matter. All or some of the layers can be stacked together and positioned adjacent to the wound. Part of the layers can be positioned aside the bioelectrical layers and not stacked on top of them.

Reference is now made to FIG. 5 schematically illustrating the components of a microelectronic dressing device, in accordance with some exemplary embodiments of the disclosed subject matter.

Two main substrates are provided to the microelectronic dressing device—the bioelectronic components 500 and the electronic device 520. The bioelectronic layer 500 comprises at least two layers, one of which is the layer that is adjacent and in intimate contact to the wound and another layer, which is gas permeable, that is provided on top of the layer that is adjacent to the wound, wherein the top layer has a perimeter that is larger than the perimeter of the lower layer.

Bioelectronic components 500 comprises a set of electronic elements that comprises sensors 502 adapted at least to sense the environment of the wound and the bioelectronic layer in which they are accommodated as well as stimulating electrodes 504 that are adapted to transfer an electrical signal and stimulate the wound tissues.

The electronic device 520 is provided with a power source 558, which is to be connected to the sensors 502 and electrodes 504 so as to activate them. Additional components in the PCB that needs power are also powered by the power source 558, however, in order to simplify the figure, the wiring is not shown herein.

A processor or CPU 552 is provided that is connected to at least the sensors 502 so as to receive the information regarding the environment of the wound. The CPU 552 is configured to manipulate the information and activate the sensors 502 and the electrodes 504. Information and signals flow between the CPU 552 and other components of the electronic device 520 while signals generated by the electrodes 504 are controlled as well as the sensors 502 in case the sensors are configured to generate signals as well.

It should be mentioned that the PCU 552 and other components of the electronic device can be incorporated as a single unit rather than separate units without limiting the scope of the present subject matter.

The electronic device 520 further provided with electronic components that facilitate the transfer and receipt of the information from and to the sensors and electrodes as well as to external devices. Other components can be selected from a group of components such as antenna 554 to transmit information, filter 556 to filter data, oscillator 560, resistors capacitors 562, memory 554 to store data, power management 564, a combination thereof and the like.

The information is transferred to external device 580 that can be in a clinic, a hospital, physician room, or any other remote place with personnel or caregivers that should be aware of the information from the sensors and of the healing progress. The external device 580 comprises an activation app 582 that is preferably a proprietary application as a computer software, health application, a combination thereof or the like that is downloadable on any electronic device such as a personal computer, a tablet, a cellphone, a terminal, a combination thereof or the like.

Optionally or alternatively, a wireless reader 584 is also provided in the external device 580.

An internet network 560 such as WiFi, Bluetooth, ethernet, a combination thereof or the like allows transfer of the information between the external device 580 and the electronic device 520.

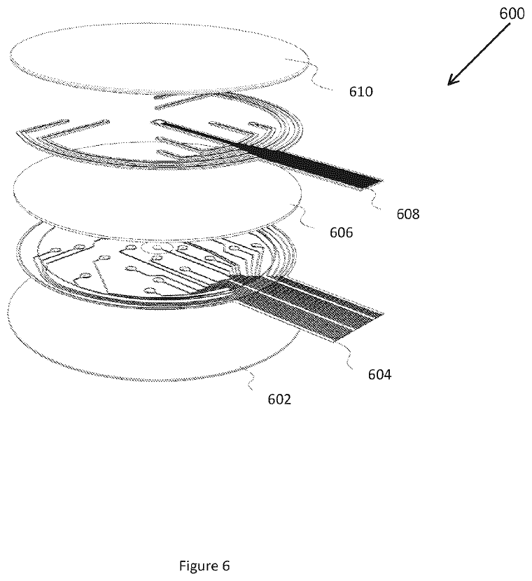

Reference is now made to FIG. 6 depicting an exploded view of a microelectronic multi-layered dressing device, in accordance with some exemplary embodiment of the disclosed subject matter.

The layers of the multi-layered dressing device 600 can be seen stacked one on top of the other while the lower-most polymer layer 602 is the layer that is in intimate contact with the wound. On top of the layer 602, a sensor layer 604 is shown. The sensors are distributed all over the layer so as to sense the whole area of the wound.

A gas permeable layer 606 is stacked on top of the sensing layer and a set of electrodes 608 is mounted on top of the gas permeable layer 606.

On top of the electrodes, the top-most layer of PCB 610 is mounted. As mentioned herein before, the PCB layer can be placed in a close location to the bioelectronic layers and not stacked on top of it.

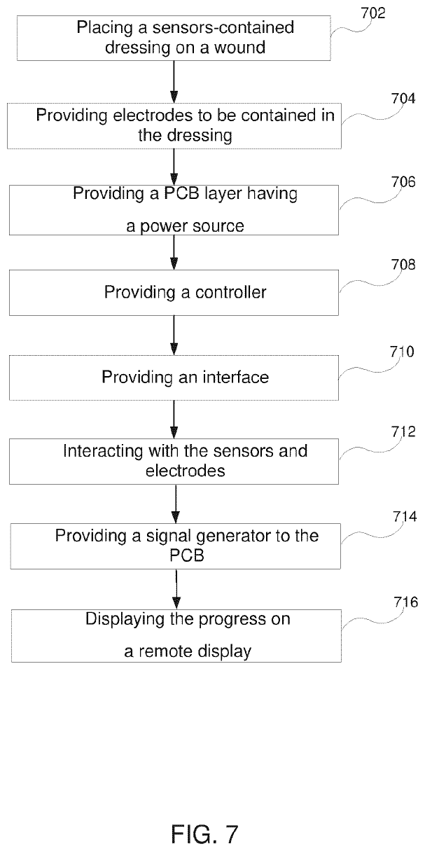

Reference is now made to FIG. 7 illustrating a flowchart diagram of a method of tracking and enhancing a wound healing process, in accordance with some exemplary embodiments of the disclosed subject matter.

By using the dressing devices described herein before, it is possible to track and closely monitor the healing process of a wound while, simultaneously, enhancing the healing process. The enhancement of the wound healing progress can be predetermined or, it can be based on the on-line results of the monitoring process.

As a first step, an adequate dressing is being provided that comprises a bioelectric layer provided with sensors. The dressing is being placed adjacent to the wound 702. At least part of the sensors should be placed adjacent to the surface of the polymeric dressing that is in contract with the wound surface, however, some of the sensors can be placed in the upper part of the bioelectric layer. The sensors can sense characteristics of the environment and at least a pH sensor is provided.

At an upper portion of the bioelectric polymer layer, a plurality of electrodes is provided 704. The electrodes can be embedded within the bioelectric layer or can be positioned on the surface of the bioelectric layer. According to a preferred embodiment, the portion of the bioelectric layer in which the electrodes are embedded or placed on its surface is a gas permeable layer. The bioelectric layer is comprised of two polymeric layers as mentioned herein before. The layer that is adjacent to the wound is provided with sensors while the layer mounted on it is a gas permeable layer that is provided with additional sensors as well as electrodes.

The placement of the dressing on the wound is arranged so that at least one of the electrodes is positioned on a portion of the bioelectric layer that is placed beyond the perimeter of the wound so that the electrode is placed on a healthy skin portion.

Optionally or alternatively, it is possible to arrange the dressing so that all electrodes are being placed on healthy skin portions rather than the areas of the dressing that are adjacent to the wound.

The next step is providing a PCB layer 706 that may be stacked on the bioelectric layer or separated from it. The PCB layer is provided a least with a power source to power all elements in the dressing that needs electrical activation such as the sensors and the electrodes.

In the next step, the PCB layer is provided with a controller 708. The controller is adapted to receive information through signals generated by the sensors and transfer instructions to the electrodes so as to activate them. The controller can have a unique program through which it controls the monitoring process of the wound as well as its healing through electrical stimulation from the electrodes. The controller can be incorporated with a processor as a single unit or can be physically separated from the processor or computing device. In any case, the controller and the computing device are communicating between themselves so as to coordinate the monitoring and enhancing healing processes, if needed.

Optionally or alternatively, a next step is providing an interface on the PCB 710. The interface allows a user or a caregiver to interact (712) with the dressing device: to receive information on the environment in contact with the wound through the sensors and transmit stimulation signals through the electrodes so as to enhance the healing progress.

Optionally or alternatively, a next step is providing a signal generator in the PCB layer 714 that allows transmitting at least one of the signals received from the sensors, signals transmitted by the electrodes, processed information from a processor in the PCB layer, instructions from the controller, a combination thereof or the like.

The transmitted information can be displayed on a remote display 716, as an example. The remote display receives the information from the computing device and/or the controller through a network such as WiFi. The display can be of a medical institute or a physician's office, as examples.

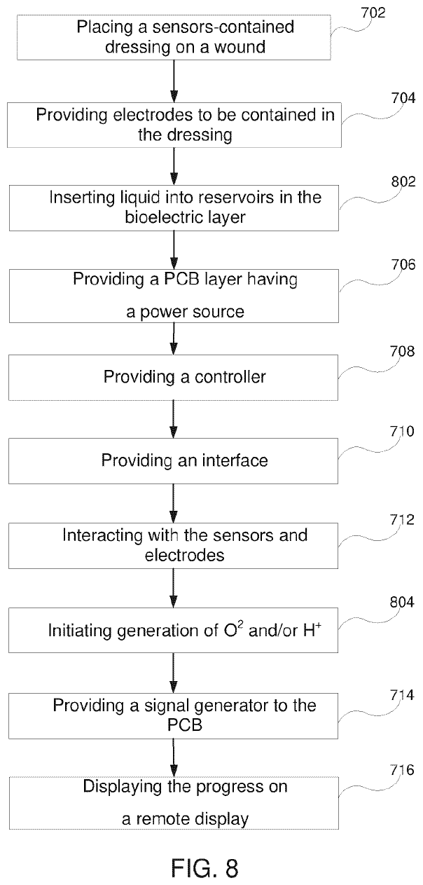

Reference is now made to FIG. 8 illustrating a flowchart diagram of a method of tracking and enhancing a wound healing process, in accordance with other exemplary embodiments of the disclosed subject matter.

In accordance with another embodiment of the present subject matter, steps 702-704 are the same as in the former method described.

Optionally or alternatively, the gas permeable layer is provided with liquid reservoirs that are filled with liquid selected from a group of liquids such as water, liquid acid solution, hydrogen peroxide, a combination thereof or the like. The liquid can be inserted within the microchannels (reservoirs) 802 in the gas permeable layer using injection or any other method. The purpose of the liquid is to allow oxygen or ionic hydrogen to permeate to the vicinity of the would so as to assist or enhance the healing process.

Generating the O₂ or H⁺ in the microchannels or reservoirs can be activated using the electrodes, as an example. Therefore, the electrodes should be controlled. Steps 706-712 of the former embodiment (FIG. 7) are the same: providing a PCB layer with power source, a controller and an interface so as to allow a user or a caregiver to interact with the sensors and electrodes.

Optionally or alternatively, the user or caregiver can activate the electrodes or the sensors so as to initiate the generation of the O₂ or H⁺804 and enhance the healing process. The activation can also be in a premeditated program that can, as an example, initiate the generation of oxygen and hydrogen in predetermined times or according to the signals received from the sensors. Alternatively, the user or the caregiver can initiate the generation of oxygen or hydrogen in the microchannels through the interface on the PCB or elsewhere according to a current situation of the wound environment.

Optionally or alternatively, the steps 714 and 716 are substantially as explained herein before.

It should be clear that multiple external devices can be provided to a system that monitors a specific dressing while a single or multiple external devices can monitor a plurality of dressings.

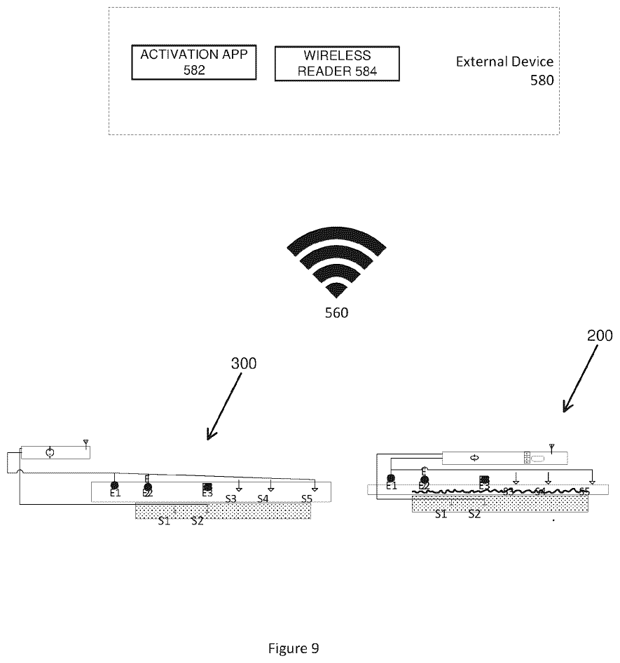

Reference is now made to FIG. 9 illustrating a block diagram of a system for tracking and enhancing wound healing process, in accordance with other exemplary embodiments of the disclosed subject matter. The system that can be used in a clinic, as an example, comprises a plurality of microelectronic multi-layered dressing devices 200 or 300, as described in regard to FIGS. 1 and 2, as examples. The multilayered dressing devices 200 or 300 can be placed on a single patient on multiple wounds or on several patients, all according to the needs of the patients in the clinic. Each dressing is provided with its own electronic device as explained herein in FIG. 5, however, in case of a single patient, it is possible that a single or more PCB layers can share some of the components such as memory capability or processor. A single external device (or a plurality in case a physician from the clinic downloads the activation app on his cellphone, as an example) receives through the network 560 information from the plurality of multilayered dressings devices 200 or 300. Any combination of dressings and external devices is possible in accordance with the present subject matter.

In order to test the effect of electrode layer provided with bioelectrodes on bacterial growth in a medium, an in-vitro experiment was performed. The inventors performed in-vitro tests using a bioelectrode with currents between 5-800 μA. An electrode layer was made of a thermoplastic polyurethane (TPU) with 8 electrode pairs. For the in-vitro experiments, all the electrodes were tested. The prevention of growth of Pseudomonas Aeruginosa applied as planktonic bacteria was tested with a concentration of 1 million cells per milliliter in Cu₂O-polysaccharide complex. The experiment was performed over a period of 48 hours in which signaling was applied on the bacterial cells. In parallel, a reference experiment was performed with the same set-up of an electrode layer; however, in the control experiment, the was no activation of any electric signaling.

After an operation of electric signaling in intervals of 1 h, 2 h, 4 h, 6 h, 8 h, 12 h, 24 h, and 48 h, the cells were diluted in dilutions of 1:10, 1:100, 1:1000, 1:10000, and 1:100000. Three drops of 5 μL per drop were placed in one line for each dilution. LB Agar plates with the dilutions were incubated for 24 hours at 37° C. (under dark conditions).

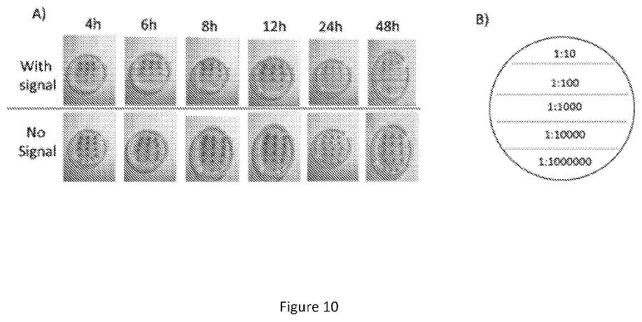

Reference is now made to FIG. 10 depicting images of the petri dishes after each of the timeslots showing the results of the in-vitro experiment made in accordance with exemplary embodiment of the disclosed subject matter.

It can be clearly seen in the left-hand side of the figure (A) that the bacterial growth is inhibited with time especially in the diluted samples in the upper row of petri dishes, where signal was applied onto the dishes. The lower row of petri dishes are the reference dishes in which signaling was not applied. Very slightly changes over time can be observed. The dilutions are indicated on the right-hand side of the figure (B).

Quantitative analysis over time was performed and the results are shown in the tables below. The results of the different dilutions that were treated with electric signaling are presented in Table 1 whereas, Table 2 shows the equivalent analysis for the reference experiments that were performed under the same conditions, but without activation of electric signals.

TABLE 1
Electric Signal Applied: Quantitative Analysis of bacterial
growth after 24 hours of incubation on petri dish.

| Dilution | | | | | | | | |
|---|---|---|---|---|---|---|---|---|
| factor | 1 h | 2 h | 4 h | 6 h | 8 h | 12 h | 24 h | 48 h |
| 10 | y | y | y | y | y | y | y | y |
| 100 | y | y | y | y | y | y | y | y |
| 1000 | y | y | y | y | y | y | y | n |
| 10000 | y | y | y | y | y | y | n | n |
| 100000 | n | n | n | n | n | n | n | n |

TABLE 2
Reference experiment without electric signal. Quantitative Analysis
of bacterial growth after 24 hours of incubation on petri dish.

| Dilution | | | | | | | | |
|---|---|---|---|---|---|---|---|---|
| factor | 1 h | 2 h | 4 h | 6 h | 8 h | 12 h | 24 h | 48 h |
| 10 | y | y | y | y | y | y | y | y |
| 100 | y | y | y | y | y | y | y | y |
| 1000 | y | y | y | y | y | y | y | y |
| 10000 | y | y | y | y | y | y | y | y |
| 100000 | n | n | y | y | y | y | y | y |

The results show that after 4 hours of applying a signal on the petri dish, a significant reduction of one dilution factor is recognized. This finding indicated that the electric signal has a bacteriostatic effect. Analyzing the samples after longer exposure to the electric signal indicates that the electric signal used has a bacteriostatic and bacteriolytic effect on the growth of the bacteria. The bacterial presence has been reduced by 2 orders of magnitude during 48 hours of operation.

To strengthen the statement, the sample after 48 hours was analyzed and prepared for quantitatively measuring the turbidity of the medium with a micro-plate reader (BioTek Instruments) at a wavelength of 600 nm. Growth inhibition was calculated from the following formula inhibitory index (%):

$$inhibitory index ( \% ) = ( 1 - \frac{{OD}_{treatment} - {OD}_{treatment - blank}}{{OD}_{control} - {OD}_{control - blank}} ) \times 100$$

where OD_treatment is the absorbance of the sample of the Cu₂O-polysaccharide complex. The OD of the solution with electric signaling was 0.1128 while the reference sample without electric signal is 0.6178. Hence, the quantitative reduction of bacterial growth has a factor of 6 while using the device of the present subject matter.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present subject matter.

1. A multi-layered dressing device to be placed on a wound having a perimeter, the multi-layered dressing device comprising:
a dressing layer to be placed on the wound, wherein the dressing layer is extended slightly beyond the perimeter;
at least one sensor in the dressing layer capable of transmitting electronic signals from a vicinity of the wound;
at least two electrodes in the dressing layer wherein one of the at least two electrodes is positioned externally to the perimeter of the wound, and wherein the at least two electrodes are capable of transmitting stimulating signals to the wound; and
a PCB layer for activating the at least one sensor and the at least two electrodes, wherein the PCB layer accommodates a computing device configured at least to receive the electronic signals and control the stimulating signals.

2. The multi-layered dressing device as claimed in claim 1, wherein the dressing layer comprises a polymer layer to be in an intimate contact with the wound, and a gas permeable layer placed on top of the polymer layer, wherein the gas permeable layer has an edge that extends beyond the perimeter of the wound.

3. The multi-layered dressing device as claimed in claim 2, wherein at least one of the at least one sensor is positioned in the polymer layer.

4. The multi-layered dressing device as claimed in claim 2, wherein the at least two electrodes are placed within the gas permeable layer.

5. The multi-layered dressing device as claimed in claim 1, wherein the at least two electrodes are placed beyond the perimeter of the wound.

6. The multi-layered dressing device as claimed in claim 2, wherein the gas permeable layer is provided with liquid reservoirs.

7. The multi-layered dressing device as claimed in claim 6, wherein the liquid reservoirs are microfluid channels that are gas permeable but liquid impermeable and wherein the microfluid channels are configured to transfer O₂ or H+ to an environment of the wound.

8. The multi-layered dressing device as claimed in claim 7, wherein the O₂ or H+ are generated within the microfluid channels.

9. The multi-layered dressing device as claimed in claim 7, wherein one of the at least two electrodes is configured to stimulate the generation of O₂ or H+ in the microfluid channels.

10. The multi-layered dressing device as claimed in claim 1, wherein the PCB layer is further provided with an interface allowing a user to interact with the computing device.

11. The multi-layered dressing device as claimed in claim 1, wherein the PCB layer is further provided with a signal generator capable of transmitting information from the computing device to an external device.

12. The multi-layered dressing device as claimed in claim 1, wherein the PCB layer is further provided with a display for displaying information gathered from the at least one sensor.

13. The multi-layered dressing device as claimed in claim 1, wherein the PCB layer is stacked on top of the dressing layer.

14. The multi-layered dressing device as claimed in claim 1, wherein the PCB layer is spaced apart from the dressing layer.

15. A method of tracking and enhancing a wound condition comprising:
providing a sensor-contained wound dressing;
placing the wound dressing on the wound, so that the wound dressing is slightly extended beyond a perimeter of the wound;
providing a layer of electrodes wherein at least one of the electrodes is positioned on an edge that will be placed beyond the perimeter of the wound;
providing a PCB layer having a power source so as to power at least the sensors and the electrodes; and
providing a controller configured to receive signals from the sensors and transmit signals through the electrodes.

16. The method as claimed in claim 15, further comprising:
providing a gas permeable layer having liquid reservoirs and stacking it on top of the sensors-contained wound dressing;
inserting liquid to within the liquid reservoirs; and
initiating generation of oxygen and/or ionic hydrogen in the liquid reservoirs using signals from the electrodes.

17. The method as claimed in claim 15, further comprising:
providing an interface in the PCB layer; and
interacting with the sensors and electrodes through the interface.

18. The method as claimed in claim 15, further comprising providing a signal generator to the PCB so as to allow transferring information to external devices.

19. The method as claimed in claim 18, wherein the external devices can be a display.

20. The method as claimed in claim 17, wherein the interface comprises a display.

21. (canceled)
